(19) United States
(12) Patent Application Publication
(10) Pub. No.: US 2025/0000681 A1
(43) Pub. Date: Jan. 2, 2025

(54) FINGER ORTHOSIS AND METHOD OF TREATMENT

(71) Applicant: Emily A. Hartnett, Denver, CO (US)

(72) Inventor: Emily A. Hartnett, Denver, CO (US)

(21) Appl. No.: 18/344,246

(22) Filed: Jun. 29, 2023

Related U.S. Application Data
(60) Provisional application No. 63/397,478, filed on Aug. 12, 2022.

(51) Int. Cl. A61F 5/01

(52) U.S. Cl. CPC A61F 5/0118

(57) ABSTRACT

The present application is directed to a Finger Orthosis and Method of Treatment which includes a finger ring orthosis and a method of non-surgical treatment techniques for finger injuries. A finger orthosis and a method of treatment for finger injuries is provided which allows for continued active even strenuous use of the injured finger while undergoing physical therapy and a rehabilitation regimen. The finger orthosis is configured as a ring, to fit onto the injured finger, and is constructed of readily available immobilization materials. The method of making the finger orthosis and the method of using the finger orthosis for treatment of injured fingers is disclosed. Results of medical testing with four individual patients illustrates the effectiveness of the finger orthosis in healing the finger injury without the need for surgical techniques, even while allowing active continued use of the patient's injured and treated finger in strenuous activities.

### FIELD OF THE INVENTION

This application relates to a finger orthosis and a method of non-surgical treatment techniques for finger injuries. More particularly, a finger orthosis and a method of treatment for finger injuries is provided which allows for continued active use of the injured finger while undergoing occupational hand therapy and a rehabilitation regimen. The finger orthosis is configured as a ring, to fit onto the injured finger, and is constructed of readily available orthotic fabrication materials. The method of making the finger orthosis and the method of using the finger orthosis for treatment of injured fingers is disclosed. Results of medical testing with four individual patients illustrates the effectiveness of the finger orthosis in healing the finger injury without the need for surgical techniques, even while allowing active continued use of the patient's injured and treated finger in strenuous activities.

### BACKGROUND OF THE INVENTION

Rock climbing is a sport which requires strength and agility. Recently, the Olympics have added rock climbing as a sport which has significantly increased the population size of climbers (Bisharat). Active rock climbers are prone to many bodily injuries, especially hand and finger injuries. There are several common injuries that climbers may experience, including: (1) tendonitis: inflammation of the tendons in the fingers, wrists, elbows, or shoulders, caused by repetitive use and overuse; (2) pulley injuries: injuries to the pulleys in the fingers, which can be caused by overuse, excessive gripping, or falling; (3) sprains and strains: overstretching or tearing of ligaments or muscles, often caused by falls or awkward movements; and (4) blisters and calluses: repeated rubbing or friction can cause painful blisters and calluses on the hands and feet.

Finger flexor pulley injuries are most commonly seen in avid rock climbers due to this community's unique upper extremity performance patterns (Zafonte, Rendulic, & Szabo, 2014). Bollen (1988) was the first to identify this common injury among rock climbers, which speaks to the novelty of available research. Evidence-based protocols are lacking since these injuries are less common within the general population and cases are often geographically dependent (Algar & Moschetto, 2018). Second annular (A2) pulley tears account for 8% of rock climbers' injuries (Zafonte, Rendulic, & Szabo, 2014). Risk of injury to the A2 pulley is highest when in a crimp grip position with eccentric loading (Miro et al., 2021). Relevant research delves into classification of pulley tears and accompanying treatment but Piper at el., (2017) found that 21% of rock climbers did not seek professional medical treatment because they felt that healthcare providers do not possess appropriate knowledge to treat climbing-related hand injuries.

What are finger flexor pulley injuries? Pulley injuries refer to the damage or injury to the pulley system in the fingers, which is responsible for holding the flexor tendons close to the phalanx. The pulleys are small, fibrous bands that act like a pulley to keep the tendons close to the bone and prevent them from bowstringing away from the finger during gripping or climbing. When the pulley is damaged, it can cause pain, swelling, and weakness in the affected finger, making it difficult to grip or hold onto objects. If not treated, the damaged pulley can incur further damage. The most common cause of pulley injuries is overuse, especially when climbers engage in repetitive gripping or climbing movements. Pulley injuries can also occur as a result of trauma, such as a fall or sudden jerk to the finger.

Pulley injuries are classified into different grades, with a grade one pulley injury as a sprain of any single pulley with no bowstringing, which is defined as a tendon phalanx distance (TPD) of less than two millimeters. Grade two is complete A4 rupture or partial A2 or A3 rupture. Grade three is complete A2 or A3 rupture, and grade four is multiple pulleys ruptured or a single pulley rupture with lumbrical muscle or collateral ligament injury involvement. Only grade IV injuries warrant surgery secondary to the lumbrical muscle involvement. Mild injuries may require rest, inactivity and a cessation of engaging in strenuous hand usage, such as rock climbing, along with conservative treatment, while more severe injuries may require surgery to repair the damaged pulley (Schöffl et al., 2003; Chang, Torriani, & Huang, 2016).

What is the A2 pulley? The A2 pulley is a specific pulley within the pulley system in the fingers, located in the bottom portion or proximal phalanx of the finger, close to the base of the finger. It is one of the most important pulleys in the finger, as it provides a significant amount of support and stability to the flexor tendon that controls the movement of the finger. The A2 pulley is particularly important for climbers, as it helps to prevent the flexor tendon from bowstringing away from the finger during gripping or climbing movements. If the A2 pulley is damaged or injured, it can cause pain, swelling, and weakness in the affected finger, making it difficult to grip or hold onto objects.

Injuries to the A2 pulley are one of the most common types of pulley injuries among climbers, and they can range in severity from mild sprains or strains to complete ruptures. Mild injuries may require conservative treatment, while more severe injuries may require surgery to repair or reconstruct the damaged pulley. Proper warming up, stretching, and using good technique can help to prevent A2 pulley injuries in climbers.

What are treatments for an A2 pulley injury? The treatment for an A2 pulley injury will depend on the severity of the injury. In the past, mild pulley injuries required rest and in some cases conservative treatment, while more severe injuries most likely required surgery to repair or reconstruct the severely damaged pulley.

Historically, conservative treatments for mild A2 pulley injuries included: (1) rest: avoiding activities that aggravate the injury and taking a break from climbing or gripping activities for a period of time; (2) ice: applying ice to the affected area can help to reduce pain and swelling; (3) compression: wrapping the finger with an elastic bandage can help to reduce swelling; (4) elevation: keeping the affected hand elevated above heart level can help to reduce swelling; and (5) physical therapy: gentle stretching and strengthening exercises can help to improve range of motion and strengthen the affected finger.

Surgical treatments for more severe A2 pulley injuries may include: (1) pulley reconstruction: this involves replacing the damaged pulley with a graft taken from elsewhere in the body; and (2) pulley repair: this involves repairing the damaged pulley using sutures or other techniques.

Recently, research has shown that not all A2 pulley injuries warrant surgery, whereas approximately twenty years ago surgery was the only medical option. Schöffl et al. (2003) developed a grading score for pulley injuries. This score was initially created to facilitate scientific evaluation and to guide therapeutic intervention but has since been adopted by most researchers and medical practitioners (Schöffl et al., 2003; Chang, Torriani, & Huang, 2016). Schöffl et al., (2003) define grade one as a sprain of any single pulley with no bowstringing (less than two millimeters of TPD). Grade two is complete A4 rupture or partial A2 or A3 rupture (Schöffl et al., 2003). Grade three is complete A2 or A3 rupture, and grade four is multiple pulleys rupture or a single pulley rupture with lumbrical or collateral ligament injury involvement (Schöffl et al., 2003). Only grade IV injuries warrant surgery secondary to the lumbrical involvement (Schöffl et al., 2003). Several other methods exist to facilitate occupation after tearing a pulley for grade I, II, or III injuries, including immobilization or rest periods, circumferential taping. H-taping, or pulley rings. A recent literature review, however, made apparent the gaps in current treatment. Schöffl et al. (2003) and Cooper & LaStayo (2020) reference periods of immobilization but an evidence-based reason as to why is not apparent despite exhaustive research. Schweizer (2000) found that it is minimally effective to apply circumferential tape for A2 pulley injuries as the tape does little to absorb tendon force on the pulley or decrease bowstringing and Crowley (2016) reflects on the lack of ability of circumferential taping to prevent further rupture of an injured pulley. Schöffl et al. (2007) found that H-tape led to a 16% decrease of the tendon phalanx distance (TPD) as compared to no tape and strength of the crimp grip improved by 13%, but there was no significant improvement for the hanging position and the tape stretches after prolonged use, thus having less effect as rock climbers continue to climb up when compared to initial application (Schöffl et al., 2007).

Schneedberger & Schweizer (2016) had participants take a break from climbing for three to four weeks, then constantly wear a Pulley Ring Orthotic for eight weeks with easy climbing using larger holds with return to normal climbing with no Pulley Ring Orthotic at week twelve. This intervention reportedly decreased TPD and thirty-eight of the forty-five rock climbers reported through a questionnaire that they had regained their previous climbing level a mean 8.8 months post-injury (Schneedberger & Schweizer, 2016). No evidence appears to explain why constant Pulley Ring Orthotic wear is necessary and why the timeline of taking a break from climbing and then climbing at an easier level is necessary.

In this respect, before explaining at least one embodiment of the Finger Orthosis and Method of Treatment in greater detail, it is to be understood that the design is not limited in its application to the details of construction and to the arrangement of the components set forth in the following description or illustrated in the drawings. The Finger Orthosis and Method of Treatment disclosed herein is capable of other embodiments and of being practiced and carried out in various ways. Also, it is to be understood that the phraseology and terminology employed herein are for the purpose of description and should not be regarded as limiting.

### SUMMARY OF THE INVENTION

The primary advantage of the Finger Orthosis and Method of Treatment is to provide a pulley ring orthotic device which will heal finger injuries while allowing for continued use of the injured finger, even in extremely strenuous activities such as rock climbing.

Another advantage of the Finger Orthosis and Method of Treatment is to provide a pulley ring orthotic device which when worn protects a finger pulley injury while allowing eccentric loading, and which has been proven to promote pulley healing.

Yet another advantage of the Finger Orthosis and Method of Treatment is to provide a pulley ring orthotic device which can be readily constructed and customized to each individual such that it fits each patient perfectly upon application, and does not restrict range of motion when worn.

A further advantage of the Finger Orthosis and Method of Treatment is to provide a pulley ring orthotic device which is configured to induce an immediate strength increase and an immediate pain reduction when donned.

Yet a further advantage of the Finger Orthosis and Method of Treatment is that finger injury patients can be evaluated for the severity of their injury and have a pulley ring orthotic device custom fabricated on the same day or even at the same practitioner's office visit, and finger injury patients are able to return to strenuous climbing activities on the same day as fabrication and application of the pulley ring orthotic.

Another advantage of the Finger Orthosis and Method of Treatment is to provide a pulley ring orthotic device which can be readily adjusted or replaced by the clinician as required for swelling and or circumferential changes to the injured finger so treated.

Yet another advantage of the Finger Orthosis and Method of Treatment is to provide a pulley ring orthotic device which includes a dorsal safety release mechanism to prevent de-gloving and or other finger and hand injuries while being worn by the patient.

These together with other advantages of the Finger Orthosis and Method of Treatment, along with the various features of novelty, which characterize the design are pointed out with particularity in the claims annexed to and forming a part of this disclosure. For a better understanding of the Finger Orthosis and Method of Treatment its operating advantages and the specific objects attained by its uses, reference should be made to the accompanying drawings and descriptive matter in which there are illustrated the preferred and alternate embodiments of the Finger Orthosis and Method of Treatment. There has thus been outlined, rather broadly, the more important features of the design in order that the detailed description thereof that follows may be better understood, and in order that the present contribution to the art may be better appreciated. There are additional features of the Finger Orthosis and Method of Treatment that will be described hereinafter, and which will form the subject matter of the claims appended hereto.

The preferred embodiment of the Finger Orthosis and Method of Treatment will include a pulley ring orthotic (PRO) device configured by utilization of thermoplastic tape, for example OrfiCast™ thermoplastic tape or the like. The construction of the PRO may include the following parameters and includes the following steps: (1) the thermoplastic tape (e.g., Orficast™ thermoplastic tape) may be heated in water in a splint pan to 140° F.; (2) the short length of thermoplastic tape may be 20% the circumference of the PRO device in order to cover the volar aspect of the adjacent phalanx; (3) the short length of thermoplastic tape should be applied to increase the pressure applied to the injured pulley at the volar aspect of the adjacent phalanx; (4) the heated thermoplastic tape may be folded to increase the strength of the tape being applied to form the PRO device; and (5) excess portions of the thermoplastic tape may extend from the PRO device once formed; these excess portions may be cut with the cut ends being heated to have at least 75% adherence to serve as a safety release while rock climbing.

Finger Orthosis and Method of Treatment primary features will include as prominent design and operational features: a method of making and applying a PRO device orthosis to a human finger to provide tendon support at an injured finger pulley, the method comprising: heating a length of thermoplastic tape; stretching the heated length of thermoplastic tape; wrapping the stretched length of thermoplastic tape about the phalanx adjacent to the injured finger pulley with ends of the thermoplastic tape being joined on the dorsal aspect of the adjacent phalanx, the ends of the thermoplastic tape being joined to define a PRO device encircling the injured finger pulley and the adjacent phalanx, the PRO device being in tension to apply pressure to the tendon to press the tendon towards the adjacent phalanx; then heating a short length of thermoplastic tape; and applying the heated short length of thermoplastic tape to the Pulley Ring Orthotic on the volar aspect of the adjacent phalanx, wherein the heated short length of thermoplastic tape is applied to increase the tension in the PRO device and pressure applied to the injured pulley.

In alternate embodiments of the Finger Orthosis and Method of Treatment, the method for making and applying the PRO device may include: further comprising folding the heated length of thermoplastic tape prior to the wrapping the stretched length of thermoplastic tape; and wherein the ends of the thermoplastic tape extend from the ring once joined, the method further comprising cutting the joined portion of the thermoplastic tape extending from the ring. And further wherein the injured finger pulley is the A2 pulley and the adjacent phalanx is the proximal phalanx; wherein the injured finger pulley is the A4 pulley and the adjacent phalanx is the middle phalanx; as well as any PRO device formed or applied to a human finger by any method of the preceding or foregoing description. It should be noted that the following PRO device and method of treatment description focuses on application to the A2 pulley and A2 pulley injuries, the PRO device and the method of treatment is equally applicable to an A4 pulley injury.

With respect to the above description then, it is to be realized that the optimum dimensional relationships for the parts of the Finger Orthosis and Method of Treatment, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present design. Therefore, the foregoing is considered as illustrative only of the principles of the Finger Orthosis and Method of Treatment. Further, since numerous modifications and changes will readily occur to those skilled in the art, it is not desired to limit the to the exact construction and operation shown and described, and accordingly, all suitable modifications and equivalents may be resorted to falling within the scope of this application.

### BRIEF DESCRIPTION OF THE DRAWINGS

The accompanying drawings, which are incorporated in and form a part of this specification, illustrate embodiments of the Finger Orthosis and Method of Treatment and together with the description, serve to explain the principles of this application.

FIG. 1 depicts Table 1 illustrating Finger Orthosis and Method of Treatment study participant demographics for each study participant.

FIG. 2 depicts Table 2 which includes Finger Orthosis and Method of Treatment study functional strength measurements for each study participant, obtained using a crimp grip method.

FIG. 3 depicts Table 3 which includes Finger Orthosis and Method of Treatment study functional strength measurements for each study participant, obtained using a weight bearing offload on a hang board method.

FIG. 4 depicts Graph 1 illustrating a graphical analysis of functional strength of each study participant over time via a crimp grip test.

FIG. 5 depicts Graph 2 illustrating a graphical analysis of functional strength of each study participant over time via a weight bearing offload test.

FIG. 6 depicts Graph 3 illustrating a graphical analysis of swelling changes over time during intervention by each study participant.

FIG. 7 depicts Graph 4 illustrating a graphical analysis of subjective functional ability of each study participant through the QuickDASH Sport outcome measure.

FIG. 8 depicts Graph 5 illustrating a graphical analysis of subjective pain reported by each study participant.

### DETAILED DESCRIPTION OF THE PREFERRED EMBODIMENTS

As required, the detailed embodiments of the present Finger Orthosis and Method of Treatment are disclosed herein, however, it is to be understood that the disclosed embodiments are merely exemplary of the design that may be embodied in various forms. Therefore, specific functional and structural details disclosed herein are not to be interpreted as limiting, but merely as basic for the claims and as a representative basis for teaching one skilled in the art to variously employ the present design in virtually any appropriately detailed structure as well as combination.

There appears to be a gap in research explaining if or why immobilization is medically necessary to treat finger pulley injuries, and there exists non-conclusive literature on which orthotic intervention is best practice. An evidence-based protocol which facilitates A2 or A4 pulley healing while promoting occupational engagement, in this case rock climbing, is needed. The following study and resulting discussion seeks to address the question: “What are the outcomes of a rock climber with a grade one, two, or three A2 pulley following a protocol which involves rock climbing with a Pulley Ring Orthotic donned for twelve weeks?”

A prospective repeated-measures case series was used to follow the healing timeline of rock climbers, all of whom climbed two to three times a week for two to three hours each time, while wearing a custom fabricated PRO. Measurements were taken weekly to monitor the line of progression of healing. A case series was used because controlled experimentation was involved with an A-B-A design, in which a period of baseline measurements (A) precedes a period of experimental intervention (B), followed by another period of monitoring (A).

The study took place at a private outpatient hand therapy clinic in Denver, Colorado, and in local rock climbing gyms. Recruitment occurred through word of mouth and through advertisements posted in the local rock climbing gyms and on the Facebook page, “Boulder Social Climb”. The State of Colorado permits direct access for occupational therapy, so no doctor referrals were needed unless the patient was using Medicare, which none of the participants involved used. Participants were thoroughly assessed using the Schöffl et al (2003) conservative classification system. Participants obtained their weekly measurements in either the clinic or in the local rock climbing gyms and had their intervention device, a custom-made Pulley Ring Orthotic fabricated and adjusted as needed.

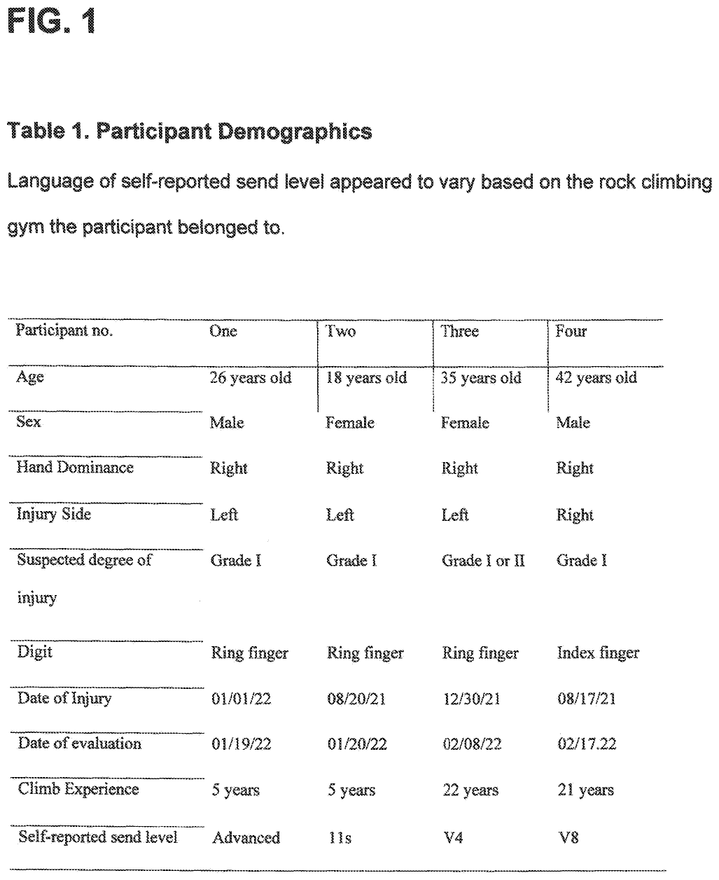

FIG. 1 depicts Table 1 which illustrates the Finger Orthosis and Method of Treatment study participant demographics for each study participant. The language of self-reported send level appeared to vary based on the rock climbing gym the participant belonged to.

The study population included self-identifying rock climbers with the following inclusion criteria: (1) a grade I to III A2 pulley injury based on guidelines set forth by Schöffl et al. (2003); (2) eighteen years old or over and able to give consent; and (3) Participates in rock climbing at least once a week.

Exclusion criteria included: (1) a pulley injury on the same finger of the opposite hand; (2) a Grade IV pulley tear (Based on guidelines set forth by Schöffl et al., 2003); and (3) potential health concerns which would raise the level of risk of injury than what is considered reasonable (e.g., active healing of an injury to other parts of the body, suspected cognitive/psychological impairment, etc.).

Four participants (two females and two males) between the ages of eighteen and forty-two (mean, 30.25 years; standard deviation, 9.07 years) participated in this study. Onset of symptoms varied from three weeks to six months. All patients sustained a grade I, II, or III injury to an A2 pulley on one digit (three ring fingers, one index) with no symptoms on contralateral digit.

Data collection occurred weekly for a 12 week period. Week one included custom Pulley Ring Orthotic fabrication. The Pulley Ring Orthotics were fabricated from three centimeters by eighteen centimeter pieces of Orficast™ Thermoplastic Tape (North Coast Medical, Morgan Hill, CA) stretched to 100% and folded in half, thus making the Orficast™ more rigid and two-ply. Edges were then cut to make the Orficast™ smooth and narrow to avoid impingement of active range of motion (AROM). The material was then wrapped around the proximal phalanx of the involved digit with the two tails being pinched together at the dorsal proximal phalanx. Full intrinsic minus, plus, and first were then assessed and adjustments were made if the Pulley Ring Orthotic caused barrier to range of motion. A two and half centimeters by two and half centimeters (adjusted per patient as needed) piece of Orficast™ was heated in the splint pan for five to ten seconds and then adhered to the inside volar aspect of the Pulley Ring Orthotic to add more pressure to the involved A2 pulley area. The dorsal tails of the ring were then cut off, and the Pulley Ring Orthotic was broken open and re-adhered using a heat gun and tweezer for eight to ten seconds.

### Intervention

In summary then, week one included custom PRO fabrication. The PROs were fabricated from three-centimeter by eighteen-centimeter pieces of Orficast™ Thermoplastic Tape (North Coast Medical, Morgan Hill, CA). Orficast™ Thermoplastic Tape was particularly suited for this study as it is a patented immobilization hybrid fabric. More particularly, Orficast™ Thermoplastic Tape is described in U.S. Pat. No. 10,081,889 issued to Cuypers et al. on Sep. 25, 2018, as a hybrid fabric comprising at least one structural fiber having a first melting temperature range and at least one thermo-formable fiber of having a second melting temperature range which is lower than the first melting temperature range to such an extent that the structural fiber does not melt in the second melting temperature range of the thermo-formable fiber. The hybrid fabric comprises (1) a first network comprising the structural fibers, (2) a second network comprising the thermo-formable fibers, the thermo-formable fibers comprising non-relaxed fibers made of a thermoplastic material, and (3) a plurality of openings between the fibers of the first and second network. The structural and thermo-formable fibers of respectively the first and second network interpenetrate one another and are connected to each other over at least part of the first and second network at a plurality of knot positions in the fabric where the structural and thermo-formable fibers are interwoven. The result is a rigid brace material which is also light and comfortable to wear, skin friendly, and provides cushioning and exhibits some breathability to permit air and moisture transport from the skin through the material, all of which significantly improves comfort to the patient and at the same time minimizes the risk to skin damaging or wounding.

In summary then, this piece of Orficast™ was stretched to 100% after being placed in a splint pan for approximately ten seconds then folded in half, thus making the Orficast™ more rigid and two-ply. Length-side edges were then cut to make the Orficast™ smooth and narrow to avoid impingement of active range of motion (AROM). The material, while still warm and pliable, was then wrapped around the proximal phalanx of the involved digit with the two tails being pinched together at the dorsal proximal phalanx.

### Safety Concerns Addressed

The dorsal aspect of the PRO was purposely weakened as a safety precaution. Sample PROs were tested three times to see at what unit of force the dorsal safety release would break. The examiner stood with both feet on a one by two board of wood which had two holes drilled in it with a cord of rope through the holes and attached to a carabiner which descended from a Tindeq Progressor 150. A Tindeq Progressor 150 is a Bluetooth dynamometer made specifically for testing climbers' strength (Tindeq). Climbers use this device to measure finger endurance, peak force, or rate of force development. Three PROs were tested by clipping each PRO (one at a time) in between two carabiners which were attached to the second end of the Tindeq. The examiner pulled upwards while standing on the board of wood. All three PROs broke at 6.9 kg, which is 67.67 N of force, according to the Tindeq.

The purpose of the dorsal safety release was to prevent the PRO from getting stuck on a rock wall and causing further injury such as degloving. Degloving, also called avulsion, is defined as a type of severe injury that happens when the top layers of your skin and tissue are ripped from the underlying muscle, connective tissue or bone. Degloving in climbers has been reported in Bienasz, G. (2021), Carpenter H. (2016) and Krishnamoorthy R. (2011). It can affect anybody part, but in climbers it is more common to deglove a finger. As a further safety measure, the dorsal safety release, if the finger swells to the point of circulation concerns, the climber could break the PRO off using the dorsal safety release and pressure from the contralateral hand and bring the broken PRO back to the examiner's clinics to have it re-adhered. For a ring to deglove a finger, the force acting on the ring is equal to the person's body weight and then multiplied by acceleration due to gravity. Therefore, the force of failure for that ring must be high to deglove a finger. A biomechanical study measured the ultimate force of failure of metal rings versus silicone rings; these measurements were 495.2 N versus 53.0 N, respectively. The force of failure of any pulley ring or like object must be a lower number, comparable to that of the silicon ring. For this reason, a plastic pulley ring enclosed via tape is not the most suitable option for avid rock climbers unless that tape will definitely break at a low force.

In summary then, the dorsal aspect of the Pulley Ring Orthotic was purposely weakened as a safety precaution. The purpose of the dorsal safety release is to prevent the Pulley Ring Orthotic from getting stuck on a rock wall and causing further injury; further, if the finger swells to the point of circulation concerns, the climber can break the Pulley Ring Orthotic off and bring it back to the clinic to have it re-adhered.

To ensure the PROs were properly fit with proper allowance of movement and effectiveness in rock climbing facilitation, participants donned their PRO and completed two tests created by this examiner and the present inventor: the crimp grip test and the weight-bearing offload on a hang board test. Therefore, to ensure the Pulley Ring Orthotic was effective, participants donned their Pulley Ring Orthotic and completed crimp grip test and the weight bearing offload test, both of which were designed and devised by the present inventor.

### Crimp Grip Test

The crimp grip test is defined as testing the measurement of a static crimp strength without involvement of the shoulder or other muscles. This test requires participants to grip a dynamometer with the handle adjusted to the most distal insertion, thus requiring a wider grip with metacarpophalangeal (MCP) extension, and then squeeze the dynamometer in a crimp grip, which is MCP joint extension with proximal interphalangeal (PIP) joint and distal interphalangeal (DIP) joint flexion. In summary then, the newly inventor devised crimp grip test required participants to grip the dynamometer with the handle adjusted to the most distal insertion, thus requiring a wider grip, and then squeezing the dynamometer in a crimp grip, which is MCP joint extension with PIP joint and DIP joint flexion. Dynamometer measurements were taken and recorded in pounds for each of the four study participants.

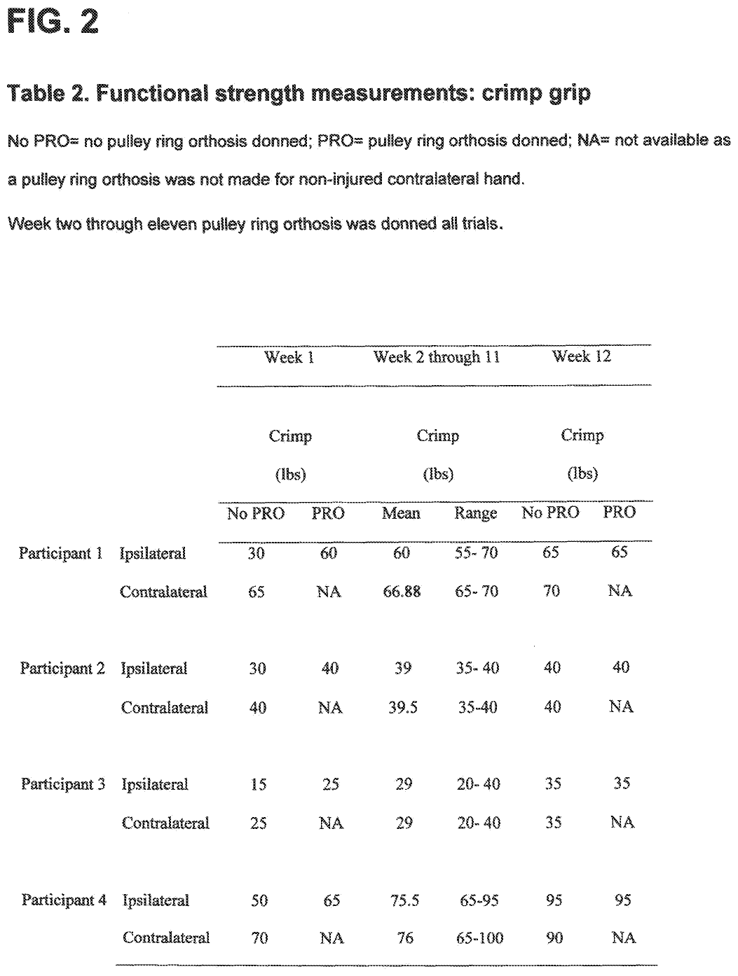

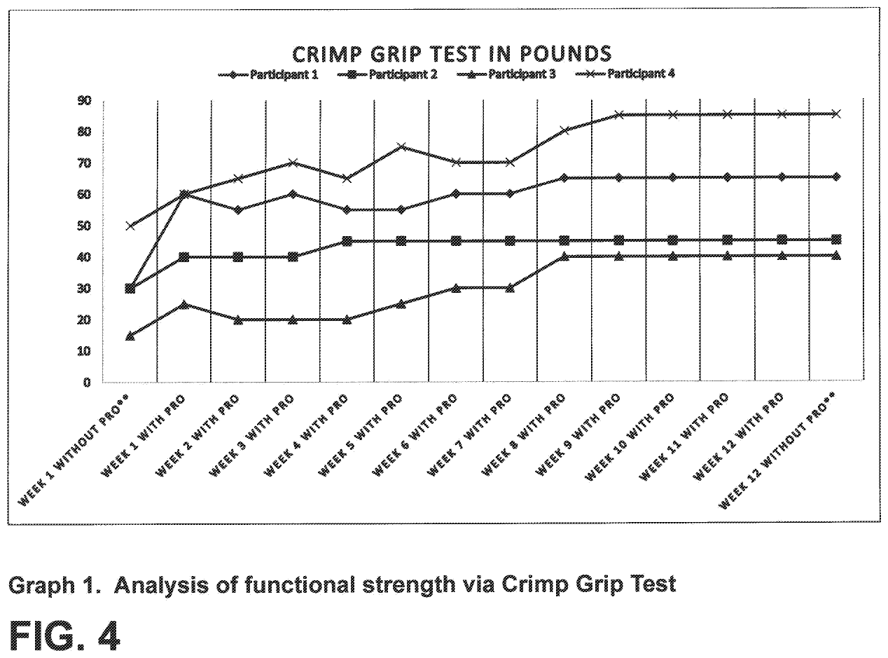

FIG. 2 depicts Table 2 which includes Finger Orthosis and Method of Treatment study functional strength measurements, in pounds, for each study participant, obtained using this crimp grip test method. FIG. 4 depicts Graph 1 which also illustrates a graphical analysis of functional strength using this newly devised crimp grip test.

### Weight Bearing Offload on a Hang Board Test

The weight-bearing offload test is defined as loading the crimp grip with involvement of other muscles and shoulder position relevancy. This test requires each participant to stand on a standard bathroom scale and then hold the hang board in a crimp grip. As stated above, it was found that H-tape led to improved static grip but not a loaded grip. This is why this study tested both a static crimp via dynamometer and loaded eccentric crimp via a scale and hang board; this case series tested both static crimp and eccentrically loaded crimp to address this gap in current knowledge. Study participants then eccentrically pulled up on the hang board while maintaining the crimp grip. The participant's offload measurement, namely, the number in pounds reading on the scale when the participant pulled up on the hang board to their maximum ability, was subtracted from their body weight to record how many pounds the participant was pulling on the hang board. In summary then, the newly inventor devised weight bearing offload test requires each participant to stand on a weight reading scale and then hold the hang board in a crimp grip. Participants then eccentrically pulled up on the hang board while maintaining the crimp grip. The participant's offload (the number on the scale when the participant pulled up on the hang board to their maximum ability) was subtracted from their body weight to record how many pounds the participant was pulling on the hang board.

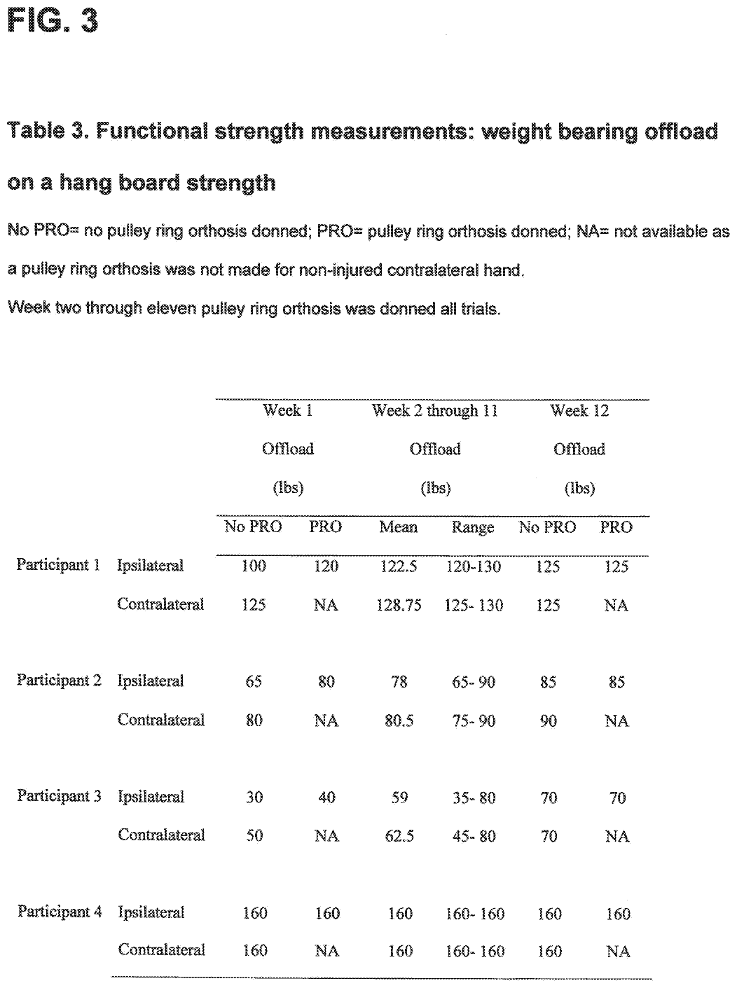

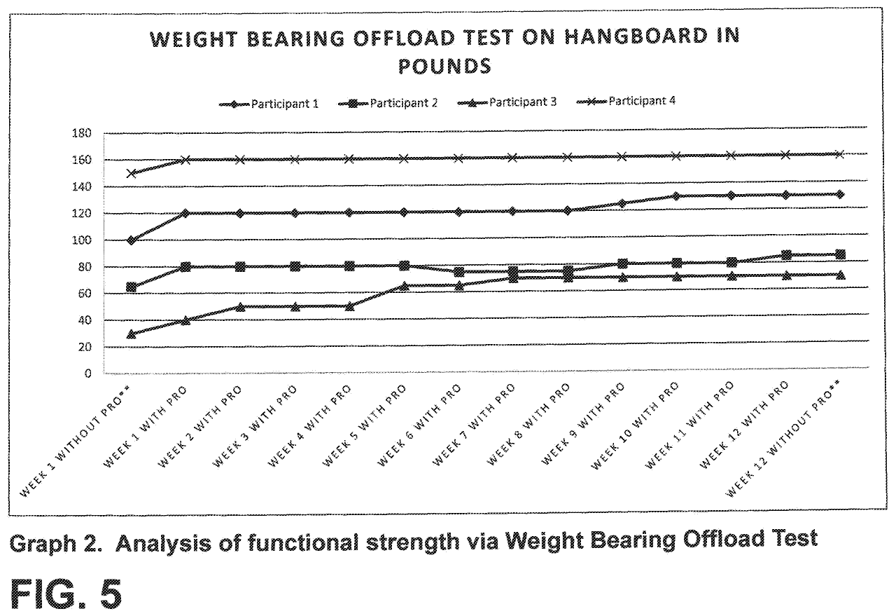

FIG. 3 depicts Table 3 which includes Finger Orthosis and Method of Treatment study functional strength measurements for each study participant, obtained using this weight bearing offload on a hang board test method. FIG. 5 depicts Graph 2 which also illustrates a graphical analysis of functional strength using this newly devised weight bearing offload test.

On day one, participants tested their involved hand twice, once with the Pulley Ring Orthotic and once without, to ensure that the Pulley Ring Orthotic was facilitating functional strength. Participants repeated both of these tests (without a Pulley Ring Orthotic) on the contralateral side. Participants also filled out the QuickDASH sport, a self-reporting questionnaire measurement tool designed to assess both symptoms and function. This outcome measure is a shortened measure of the 30 item Disabilities of the Arm, Shoulder and Hand (DASH) questionnaire (see below); four items are used instead to subjectively assess symptoms within the occupation, in this case rock climbing (Solway et al., 2006). These measurements, along with grip strength, circumferential swelling of the proximal phalanx, range of motion, and pain were taken weekly for twelve weeks and compared to the contralateral side. Participants were also asked to track hours climbed each and level climbed each week. Due to climbing grades varying geographically, inside versus outside, and from gym to gym, patients were asked to identify a send level. Send level is defined as the grade the climber typically can complete without losing grip, falling into, or suspending on their gear and will be recorded per participant. Each week, participants documented + if they averaged above send level (thus climbing “hard”), = if they climbed at their send level, or − if they climbed levels below their self-identified send levels (thus climbing “easy”).

### Data Analysis

PRO intervention success was measured through return to function, which was expressed through the QuickDASH-Sport scores, pain, and functional strength including both crimp test measurements and eccentric offload test measurements on a hang board, as well as maintenance of an active climbing schedule (frequency) for each of the four study participants. The Disabilities of the Arm, Shoulder and Hand (DASH) questionnaire is a 30-item questionnaire that looks at the ability of a patient to perform certain upper extremity activities. This questionnaire is a self-report questionnaire that patients can rate difficulty and interference with daily life on a 5 point Likert scale. The DASH has been translated in many different languages and has demonstrated to be a valid and reliable questionnaire for a variety of upper extremity disorders.

The QuickDASH is an abbreviated version of the original DASH outcome measure. In comparison to the original 30 item DASH outcome measure, the QuickDASH only contains 11 items. It is a questionnaire that measures an individual's ability to complete tasks, absorb forces, and severity of symptoms. The QuickDASH tool uses a 5-point Likert scale from which the patient can select an appropriate number corresponding to his/her severity/function level. The intended population for the DASH is any person with one or more upper extremity musculoskeletal disorders. The QuickDASH outcome measure is designed to be used on patients presenting with one or more disabilities of the arm, shoulder, and hand.

Some examples of the DASH questions are: (1) How would you rate your ability to do the following tasks 1-5 (1 being no difficulty, 5 being unable); (2) Place an object on a shelf above your head; (3) Use a knife to cut food; (4) Wash your back, etc. The latter part of the questionnaire rates your symptoms and impact on work and sport using a similar scale.

The other measurements as described above were recorded throughout the 12 week intervention study. The total percentage of improvement was measured on week one and twelve by comparing pain and functional strength measurements with Pulley Ring Orthotic doffed versus donned. This percentage was monitored with Pulley Ring Orthotic donned from there on out, and finally compared again at week twelve, as mentioned.

### Ethics/Trustworthiness

This intervention approach is based on various studies that have shown efficacy in conservative treatment of Grades I, II, and III A2 pulley tears (Schöffl et al., 2003; Cooper & LaStayo, 2020; Crowley, 2016; Algar & Moschetto, 2017; Schöffl et al., 2007; Schneedberger & Schweizer; 2016; Schweizer, 2000). Patients were offered treatment for symptom relief including ultrasound, manual techniques, and laser to complement the intervention study. This researcher and named inventor obtained institutional review board (IRB) approval prior to initiating this case series study and all four participants completed informed consent forms.

### Results

### Participant Demographics

FIG. 1 depicts Table 1 which illustrates the demographics pertaining to each study participant and his/her pulley injury. Based on patient report and testing summarized above, study participants one, two, and four all appeared to have a sprain of only one pulley, the A2, with no bowstringing or lumbricals involvement thus were considered grade one pulley injuries (Chang, Torriani, & Huang, 2016). Participant three reported consistent pain at the area of the A4 pulley with palpation and during climbing with no bowstringing or lumbricals involvement. This pain, which lessened over the twelve week intervention period, suggests a grade two pulley injury (Chang, Torriani, & Huang, 2016).

### Main Findings

FIG. 2 depicts Table 2 which illustrates a graphical analysis of functional strength of each of the four study participants over time via the crimp grip test previously described. The crimp grip test was measured in pounds for each study participant through a 12 week period of time and the results plotted. Week one and twelve were tested and measured without the PRO donned.

FIG. 3 depicts Table 3 which illustrates a tabular analysis of functional strength of each of the four study participants over time via the weight bearing offload test as previously described. The weight bearing offload test was measured in pounds for each participant through a 12 week period of time and the results plotted. Week one and twelve were tested and measured with and without the PRO donned. Both ipsilateral and contralateral measurements were taken for each study participant. For weeks 2 through 11 a mean and range in pounds were calculated.

FIG. 4 depicts Graph 1 which illustrates a graphical analysis of the functional strength of each study participant using the crimp grip test as previously described. The crimp grip test measured functional strength in pounds over a 12 week period. Week 1 and week 12 were measured with and without a PRO donned. In all of the four study participants, it can be clearly seen that functional strength measurements increased immediately in week 1 with the PRO donned. Functional strength continued to increase slightly until week 9 when each participant's functional strength leveled off through week 12.

FIG. 5 depicts Graph 2 which illustrates a graphical analysis of the functional strength of each study participant using the weight bearing offload test, using a scale and hang board, as previously described. The weight bearing offload test measured functional strength in pounds over a 12 week period. Week 1 and week 12 were measured with and without a PRO donned. In all of the four study participants, it can be clearly seen that functional strength measurements increased immediately in week 1 with the PRO donned. Functional strength continued to increase through week 12.

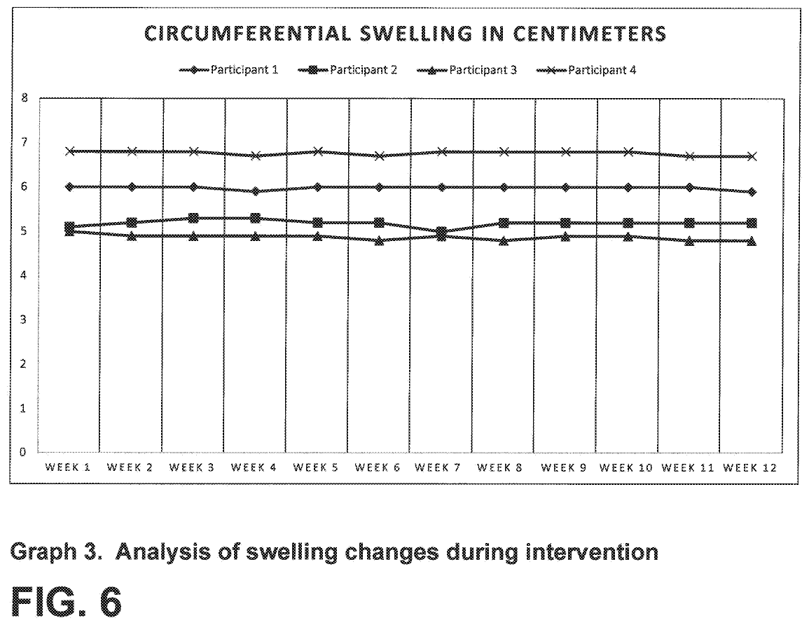

FIG. 6 depicts Graph 3 which illustrates a graphical analysis of swelling changes over time during intervention by each of the four study participants. Circumferential swelling changes were measured in centimeters over a period of 12 weeks during intervention in the study.

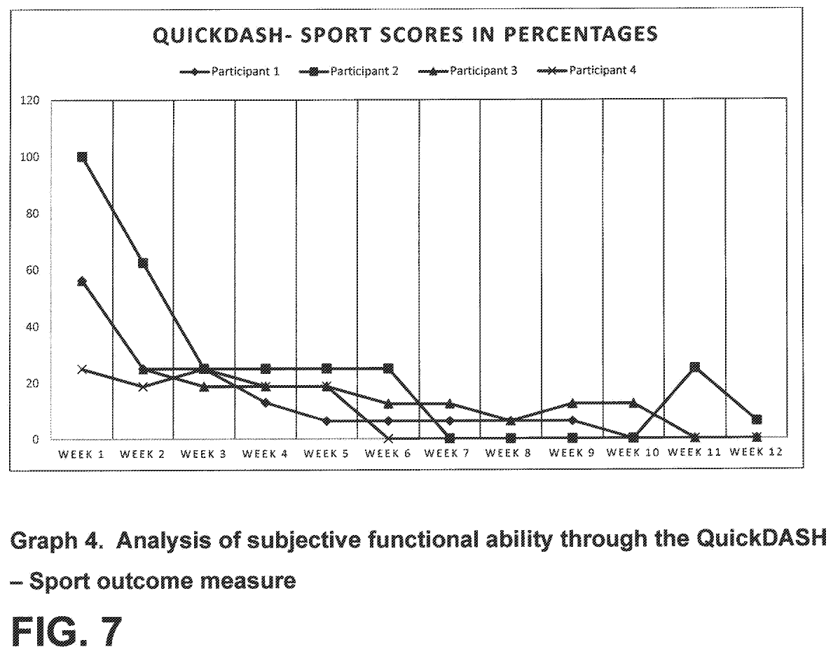

FIG. 7 depicts Graph 4 which illustrates a graphical analysis of subjective functional ability of each of the four study participants through the QuickDASH Sport outcome measure. QuickDASH Sport Scores were measured in percentages each week during the 12 week study. This resulted in a measurement indication of the subjective functional ability for each study participant in each week.

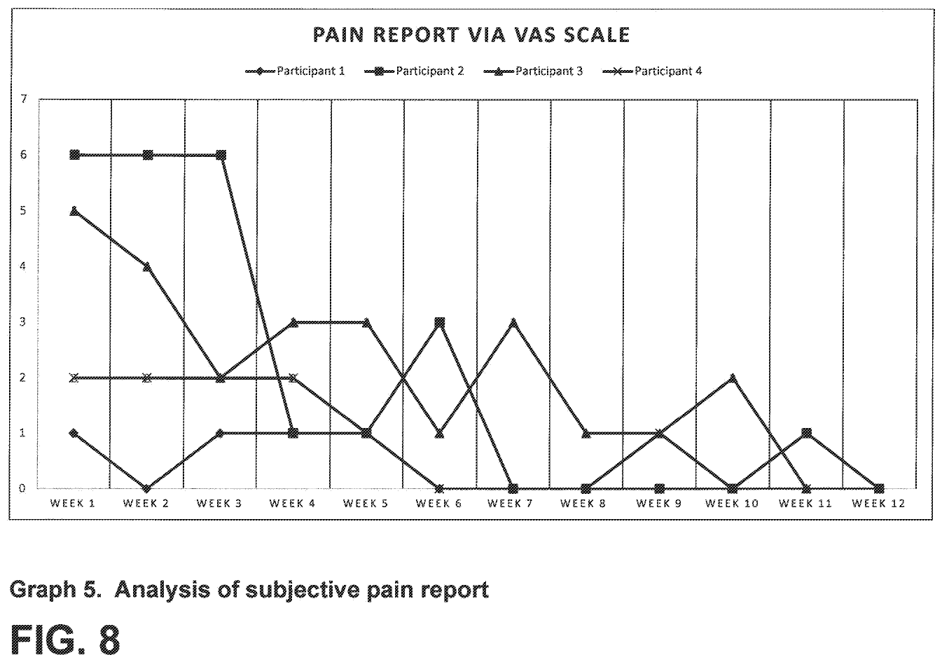

FIG. 8 depicts Graph 5 illustrating a graphical analysis of subjective pain reported by each of the four study participants. Subjective pain was reported via the VAS Scale for each week in the 12 week study.

### Example 1—Study Participant 1

On Jan. 19, 2022, a self-identified rock climber, hereby identified as participant one, reported hearing a “pop” during a crimp hold two and a half weeks prior to measurement day. He self-diagnosed a pulley injury and had not climbed since, thus had been off climbing for two and a half weeks. He was unable to crimp on the dynamometer or hang-board without immediate 4/10 pain at the area of his A2 pulley of D4 non-dominant hand. With his custom Pulley Ring Orthotic donned, his crimp on the dynamometer increased from thirty pounds to sixty pounds with 1/10 pain upon release. His opposite hand measured at sixty-five pounds. His eccentric offload on the hang board increased from 100 pounds to 120 pounds. His opposite hand measured at 125 pounds. he was able to offload within ten pounds on 40 mm hang-board ledge when compared to her dominant hand. Participant one reported 1/10 pain each trial with Pulley Ring Orthotic donned. These functional strength measurements stayed fairly consistent over the twelve-week intervention timeline, with QuickDASH score reducing overtime, see the results as mentioned above and in FIGS. 2-3 depicting Tables 2-3, as well as FIGS. 4-8 depicting Graphs 1-5. Week three participant one's Pulley Ring Orthotic was re-fabricated as it broke at the end of a climbing session. Participant one verbalized that he believed it broke because “it was stuck on [his] finger and he pulled too hard to get it off.” Measurements with the new Pulley Ring Orthotic donned were comparable to the previous week (thus original Pulley Ring Orthotic) and contralateral hand, deeming it appropriate to continue the study with a new Pulley Ring Orthotic. The new Pulley Ring Orthotic was comparable in measurements.

During week four and five participant one verbally reported 4/10 forearm pain which appeared to affect strength. Pain in involved finger did not change during testing or climbing, and objective data returned to comparative measures when this unrelated pain subsided by week six. Week seven and ten strength measurements were not taken because participant one was travelling and sick, respectively, and was thus not available for measurements but was able to complete subjective measurements via text message. Participant one reported he climbed 100% of the time with his custom Pulley Ring Orthotic donned, he did not forget it once. Week twelve was the first time that participant one completed testing with the Pulley Ring Orthotic both donned and doffed since week one.

As presented in the results as mentioned above and in FIGS. 2-3 depicting Tables 2-3, as well as FIGS. 4-8 depicting Graphs 1-5, objective data with Pulley Ring Orthotic donned matched objective data with Pulley Ring Orthotic doffed. Participant one reported he wore his Pulley Ring Orthotic while lifting in the gym and while rock climbing, and at no other times. Participant one reported pros of climbing with his Pulley Ring Orthotic for twelve weeks included: “reduced anxiety about tearing his pulley again or climbing too hard on it, felt stronger with it, and easy to get on and off”. Participant one reported cons of climbing with his Pulley Ring Orthotic for twelve weeks included: “easy to lose, it broke one time, it pinched the skin where the finger and hand meet but I built a callus or could tape it”. FIGS. 2-3 depicting Tables 2-3, as well as FIGS. 4-8 depicting Graphs 1-5 aligns with participant one's functional gains. These tabular and graphical date represent crimp strength in pounds on week one, first with no Pulley Ring Orthotic donned, followed by Pulley Ring Orthotic donned, followed by the average between weeks two and eleven, followed by week twelve with Pulley Ring Orthotic, and lastly week twelve with no Pulley Ring Orthotic. The resulting data collected represents weight bearing off load in pounds following the same timeline. This is clear evidence showing the positive correlation that Pulley Ring Orthotic application had on functional strength for study participant 1.

### Example 2—Study Participant 2

On Jan. 20, 2022 a self-identified rock climber, hereby identified as participant two, had a four month old injury she thought would “just heal” due to her age (eighteen) and continued to climb “through the pain”. She noticed an immediate decrease in pain, from 8/10 to 6/10, with Pulley Ring Orthotic donned. Her functional strength improved as well, similar to participant one and as depicted in Table 2. Participant two wore her Pulley Ring Orthotic for rock climbing only, and did not have it on during other daily occupations. This participant, however, was diagnosed with a concussion at the end of week six and was told by a physician, unrelated to this study, that she could not climb for the next four weeks. She was able to continue completing measurements, but her pain and QuickDASH scores were lower than expected likely due to not climbing. Week twelve participant two still had mild pain, 1/10, and all other measurements were within normal limits. It is hypothesized that the 1/10 with and without the ring donned was from returning to climb and was typical soreness associated with return to activity. Participant two returned to climb without the Pulley Ring Orthotic at week twelve and reported gradual reduction of residual pain and gradual return to send level. Participant two reported pros of her ring included protection and less pain, Cons were “the pain did not go away immediately.” FIGS. 2-3 depicting Tables 2-3, as well as FIGS. 4-8 depicting Graphs 1-5 aligns with participant one's functional gains and is formatted in the same manner as participant one, above. This demonstrates the positive correlation that the Pulley Ring Orthotic had on functional strength for study participant 2.

### Example 3—Study Participant 3

On Feb. 8, 2022 a self-identified rock climber, hereby identified as participant three, reported that she had a pulley injury “that was not healing because it is cold outside and [she has] poor circulation.” She was concerned she rested it for thirty days and saw no change when she attempted to return to climb. She had injured a pulley on a different finger ten years ago, and reports she did not do any form of intervention for besides take time off of climbing, she does not remember how long she took off. Participant three completed the same testing as participant one and two, and her numbers also improved immediately, as seen in Table 3. Participant three wore her ring 100% of the time throughout the twelve weeks, and was concerned with pain near her A4 pulley so taped it circumferentially for comfort. Pain in this area gradually reduced to zero out of ten by the end of the twelve week intervention. Participant two wore her Pulley Ring Orthotic for rock climbing only, and did not have it on during other daily occupations. Her reported pros includes “it works” and cons include “it gets stuck in cracks outside so I have had to avoid them.” FIGS. 2-3 depicting Tables 2-3, as well as FIGS. 4-8 depicting Graphs 1-5 aligns with participant one and two's functional gains and is formatted in the same manner as participant one and two, above. This demonstrates the positive correlation that the Pulley Ring Orthotic had on functional strength for study participant 3.

### Example 4—Study Participant 4

On Feb. 17, 2022 a self-identified rock climber, hereby identified as participant four, came in with a five-month history of pain at the area of his dominant index A2 pulley, which like the other participants, lessened when the Pulley Ring Orthotic was donned. This participant reports he had other pulley injuries in the past but he, “climbed through the pain because [he] was young.” When participant four completed measurements during week two, it was noted that his strength was reduced, pain was high, and swelling increased. The examiner thus made a new Pulley Ring Orthotic, as the first one was no longer tight enough due to the decrease in swelling. Once the ring was made smaller, pain and strength immediately returned to almost match the previous week and contralateral hand. This participant is the only one who did not wear his ring 100% of the intervention twelve week period due to forgetting his ring on two occasions. During week three, he lost the ring completely and it was re-fabricated and re-tested in the same fashion as participant one. During week six, participant four forgot his ring at home and climbed below send level only without his ring. Pain was the only measurement which appeared to change either time despite climbing without the Pulley Ring Orthotic. After week six, the patient self-directedly donned his Pulley Ring Orthotic at all times except for sleep. He reports he did this to facilitate misplacing the ring again. Participant four regards pros as feeling stronger with the Pulley Ring Orthotic donned and cons as the thickness of the orthotic causing rubbing on adjacent finger and overall personal irritation (in regard to mentality, skin appeared clean and non-aggravated each week). FIGS. 2-3 depicting Tables 2-3, as well as FIGS. 4-8 depicting Graphs 1-5 aligns with participant one, two and three's functional gains and is formatted in the same manner as participant one, two and three, above. This demonstrates the positive correlation that the Pulley Ring Orthotic had on functional strength for study participant 4.

### Conclusive Evidence

Referring now to the tabular and graphical results data illustrated in FIGS. 2-3 depicting Tables 2-3, as well as FIGS. 4-8 depicting Graphs 1-5 clearly demonstrates the positive correlation that the Pulley Ring Orthotic had on functional strength, showing increases in functional strength in each of the four study participants as measured by both the newly devised crimp grip test and the newly devised weight offload on an hang board test. Additionally, the collected data evidence shows that swelling in the injured finger during intervention as treated by the present Finger Orthosis and Method of Treatment did not significantly change. Furthermore, the data evidence demonstrates an overall decrease in pain with a concomitant decrease in QuickDASH sport outcomes subjective functional ability scores, which translates into greater functional ability with less pain involved in daily activities. All of these positive results were accomplished without a cessation of activities, including very strenuous activities such as rock climbing. All four study participants continued rock climbing while undergoing the Finger Orthosis and Method of Treatment of the present invention, throughout the 12 week study period.

### Discussion

### Interpretation of Results

As seen in figures one through four, Pulley Ring Orthotic application resulted in an immediate peak in functional strength which stayed fairly consistent until week twelve, where 100% of participants had the same strength with Pulley Ring Orthotic both donned and doffed, representing ligamentous healing despite climbing while healing. Thus, with involved structures protected, it is safe to climb while a low grade pulley injury is healing.

### Relationship to Prior Literature

Previous research has varying inclusion criteria, duration, and frequencies of immobilization (Cooper & LaStayo, 2020; Algar & Moschetto, 2018; Crowley, 2016). Current literature lacks evidence whether or not a rest and immobilization period is absolutely necessary before putting a patient at risk of occupational deprivation. Population needs include engagement in occupation, and the gap in research was putting this need at risk because there was no evidence based protocol for return to climb post-A2 pulley injury. This case series attempts to fill in these gaps and suggests that climbing while injured with custom-fit protection produces a healed ligament without occupational deprivation.

### Implications of Practice

Facilitating engagement in occupation to improve quality of life while promoting healing is a theme of occupational therapy. Having an orthotic device to fabricate for patients is a tool occupational therapists can use to both treat an injured anatomical structure while promoting engagement in daily occupations. Members of the rock climbing community participate in rock climbing as their work, play, leisure, or social participation occupation (American Occupational Therapy Association [AOTA], 2014a).

### Limitations

Limitations of this research included the small population size. Recruitment occurred in the winter and it was difficult to find participants willing to commit to the weekly check ins for twelve consecutive weeks. Further, grade of injury was assessed without imaging technology which would have confirmed the suspected grade of pulley injury.

Limitations of wearing a Pulley Ring Orthotic while climbing includes not being able to crack climb, having to stop if a Pulley Ring Orthotic is lost or forgotten or broken, and the discomfort of the ring hitting the distal palm of the hand.

Future research should include more participants. Ideally an imaging ultrasound would also be utilized to confirm the suspected grades of the pulley injuries. An addition of a quality of life assessment or measure is also recommended, as all four participants made statements implying that quality of life improved due to being allowed to climb while healing, but this was not objectively measured. A second ring should also be made for each participant so that they could have continued to climb if a ring broke during a climbing session.

### SUMMARY OF THE INVENTION

A method is provided herein of applying an orthosis to a human finger to provide tendon support at an injured finger pulley (A2, A4), the method including: heating a length of thermoplastic tape; stretching the heated length of thermoplastic tape; wrapping the stretched length of thermoplastic tape about the phalanx adjacent to the injured finger pulley with ends of the thermoplastic tape being joined on the dorsal aspect of the adjacent phalanx, the ends of the thermoplastic tape being joined to define a Pulley Ring Orthotic encircling the injured finger pulley and the adjacent phalanx, the Pulley Ring Orthotic being in tension to apply pressure to the tendon to press the tendon towards the adjacent phalanx; heating a short length of thermoplastic tape; applying the heated short length of thermoplastic tape to Pulley Ring Orthotic on the volar aspect of the adjacent phalanx, wherein the heated short length of thermoplastic tape is applied to increase the tension in the Pulley Ring Orthotic and pressure applied to the injured pulley. With the injured finger pulley being the A2 pulley, the adjacent phalanx is the proximal phalanx, while with the injured finger pulley being the A4 pulley, the adjacent phalanx is the middle phalanx.

The preferred embodiment of the Finger Orthosis and Method of Treatment will include a pulley ring orthotic (PRO) device configured by utilization of thermoplastic tape, for example OrfiCast™ thermoplastic tape or the like. The construction of the PRO may include the following parameters and includes the following steps: (1) the thermoplastic tape (e.g., Orficast™ thermoplastic tape) may be heated to 140° F.; (2) the short length of thermoplastic tape may be 20% the circumference of the PRO device in order to cover the volar aspect of the adjacent phalanx; (3) the short length of thermoplastic tape should be applied to increase the pressure applied to the injured pulley at the volar aspect of the adjacent phalanx; (4) the heated thermoplastic tape may be folded to increase the strength of the tape being applied to form the PRO device; and (5) excess portions of the thermoplastic tape may extend from the PRO device once formed; these excess portions may be cut with the cut ends being heated to have at least 75% adherence to serve as a safety release while rock climbing.

Finger Orthosis and Method of Treatment primary features will include as prominent design and operational features: a method of making and applying a PRO device orthosis to a human finger to provide tendon support at an injured finger pulley, the method comprising: heating a length of thermoplastic tape; stretching the heated length of thermoplastic tape; wrapping the stretched length of thermoplastic tape about the phalanx adjacent to the injured finger pulley with ends of the thermoplastic tape being joined on the dorsal aspect of the adjacent phalanx, the ends of the thermoplastic tape being joined to define a PRO device encircling the injured finger pulley and the adjacent phalanx, the PRO device being in tension to apply pressure to the tendon to press the tendon towards the adjacent phalanx; then heating a short length of thermoplastic tape; and applying the heated short length of thermoplastic tape to the Pulley Ring Orthotic on the volar aspect of the adjacent phalanx, wherein the heated short length of thermoplastic tape is applied to increase the tension in the PRO device and pressure applied to the injured pulley.

In conclusion, the study showed that the following advantages were attained:
1. No interrupted climbing, users can continue rock climbing while healing with the PRO;
2. The PRO has a dorsal safety release to prevent de-gloving;
3. The PRO is custom-made so it fits each patient perfectly;
4. The PRO design does not limit range of motion (ROM) when worn;
5. All subjects demonstrated full healing of the involved pulley after wearing the PRO with continued strenuous activity for 12 weeks;
6. The PRO protects the pulley injury while allowing eccentric loading, which has been proven to promote pulley healing;
7. A demonstrated immediate strength increase (when tested in crimp position) upon donning the PRO;
8. A demonstrated immediate pain reduction (when loading the crimp position) upon donning the PRO;
9. Patients can be evaluated for the injury and have their PRO fabricated on the same day; and
10. The PRO can be adjusted by the clinician as needed for swelling/circumferential changes

The Finger Orthosis and Method of Treatment shown in the drawings and described in detail herein disclose arrangements of elements of particular construction and configuration for illustrating preferred embodiments of structure and method of operation of the present application. It is to be understood, however, that elements of different construction and configuration and other arrangements thereof, other than those illustrated and described may be employed for providing the Finger Orthosis and Method of Treatment in accordance with the spirit of this disclosure, and such changes, alternations and modifications as would occur to those skilled in the art are considered to be within the scope of this design as broadly defined in the appended claims.

While certain embodiments of the inventions have been described, these embodiments have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. For example, one portion of one of the embodiments described herein can be substituted for another portion in another embodiment described herein. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y. and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

Further, the purpose of the foregoing abstract is to enable the U.S. Patent and Trademark Office, foreign patent offices worldwide and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

I claim:

1. A finger orthosis, comprising:
(a) a length of thermoplastic tape having two ends formed into a ring by a thermal joining connection of said two ends; and
(b) a heated short length of thermoplastic tape applied to the pulley ring orthotic on the volar aspect of the adjacent phalanx;
wherein said length of thermoplastic tape is configured into a pulley ring orthotic to be applied to a patient's injured finger, whereby the length of said thermoplastic tape is predetermined to result in a diameter ring size to fit each individual patient's injured finger being so applied, and said applied heated short length of thermoplastic tape to the pulley ring orthotic on the volar aspect of the adjacent phalanx increases the pressure applied to the injured pulley.

2. The finger orthosis according to claim 1, wherein said length of thermoplastic tape having two ends is folded before being formed into a ring by a thermal joining connection of said two ends.

3. The finger orthosis according to claim 1, wherein said configured pulley ring orthotic includes a dorsal safety release.

4. The finger orthosis according to claim 1, wherein said configured pulley ring orthotic when accurately fitted to each patient's injured finger does not limit finger range of motion and active range of motion.

5. The finger orthosis according to claim 1, wherein said configured pulley ring orthotic allows healing of an injured finger pulley while using the injured finger and engaging in strenuous activity.

6. The finger orthosis according to claim 1, wherein said patient's injured finger includes an injured finger pulley.

7. The finger orthosis according to claim 6, wherein said injured finger pulley includes the A2 pulley and the adjacent phalanx is the proximal phalanx.

8. The finger orthosis according to claim 6, wherein said wherein said injured finger pulley includes the A4 pulley and the adjacent phalanx is the middle phalanx.

9. A method of making a finger orthosis, comprising the steps of:
(a) providing a length of thermoplastic tape having two ends;
(b) forming said length of thermoplastic tape into a ring by bringing said two ends together;
(c) thermally joining said two ends; and
(d) applying a heated short length of thermoplastic tape to the pulley ring orthotic on the volar aspect of the adjacent phalanx;
wherein said length of thermoplastic tape is configured into a pulley ring orthotic to be applied to a patient's injured finger, whereby the length of said thermoplastic tape is predetermined to result in a diameter ring size to fit each individual patient's injured finger being so applied, and said applied heated short length of thermoplastic tape to the pulley ring orthotic on the volar aspect of the adjacent phalanx increases the pressure applied to the injured pulley.

10. The method for making a finger orthosis according to claim 9, wherein said length of thermoplastic tape having two ends is folded before being formed into a ring by a thermal joining connection of said two ends.

11. The method for making a finger orthosis according to claim 9, wherein said configured pulley ring orthotic includes a dorsal safety release.

12. The method for making a finger orthosis according to claim 9, wherein said configured pulley ring orthotic when accurately fitted to each patient's injured finger does not limit finger range of motion and active range of motion.

13. The method for making a finger orthosis according to claim 9, wherein said configured pulley ring orthotic allows healing of an injured finger pulley while using the injured finger and engaging in strenuous activity.

14. The method for making a finger orthosis according to claim 9, wherein said patient's injured finger includes an injured finger pulley.

15. The method for making a finger orthosis according to claim 14, wherein said injured finger pulley includes the A2 pulley and the adjacent phalanx is the proximal phalanx.

16. The method for making a finger orthosis according to claim 14, wherein said wherein said injured finger pulley includes the A4 pulley and the adjacent phalanx is the middle phalanx.

17. A method of treating a finger injury using a finger orthosis, comprising the steps of:
(a) applying an orthosis to a human finger to provide tendon support at an injured finger pulley, the method comprising the steps of:
(i) providing a length of thermoplastic tape;
(ii) heating said length of thermoplastic tape;
(iii) stretching said heated length of thermoplastic tape;
(iv) wrapping the stretched length of thermoplastic tape about the phalanx adjacent to the injured finger pulley with ends of the thermoplastic tape being joined on the dorsal aspect of the adjacent phalanx, the ends of the thermoplastic tape being joined to define a pulley ring orthotic encircling the injured finger pulley and the adjacent phalanx, the pulley ring orthotic being in tension to apply pressure to the tendon to press the tendon towards the adjacent phalanx;
(v) heating a short length of thermoplastic tape; and
(vi) applying the heated short length of thermoplastic tape to the pulley ring orthotic on the volar aspect of the adjacent phalanx;
wherein the heated short length of thermoplastic tape is applied to increase the tension in the configured pulley ring orthotic, and thereby increase the pressure applied to the injured pulley.

18. The method of treating a finger injury using a finger orthosis according to claim 17, wherein said configured pulley ring orthotic when accurately fitted to each patient's injured finger does not limit finger range of motion and active range of motion.

19. The method of treating a finger injury using a finger orthosis according to claim 17, wherein said configured pulley ring orthotic allows healing of an injured finger pulley while using the injured finger and engaging in strenuous activity.

20. The method of treating a finger injury using a finger orthosis according to claim 17, wherein said configured pulley ring orthotic includes a dorsal safety release.
